(19) United States
(12) Patent Application Publication
(10) Pub. No.: US 2008/0243217 A1
(43) Pub. Date: Oct. 2, 2008

(54) CARDIAC STIMULATION APPARATUS

(76) Inventor: Michael Peter Wildon, Chateauneaf de Grasse (FR)

Correspondence Address: MERCHANT & GOULD PC, P.O. BOX 2903, MINNEAPOLIS, MN, 55402-0903, US

(21) Appl. No.: 11/937,308

(22) Filed: Nov. 8, 2007

Related U.S. Application Data
(63) Continuation-in-part of application No. 10/297,189, filed on Jul. 11, 2003, now abandoned.

(30) Foreign Application Priority Data
May 30, 2000 (AU) PQ 7884

(51) Int. Cl. A61N 1/00; A61B 5/04

(52) U.S. Cl. 607/116; 607/152; 600/391; 607/115

(57) ABSTRACT

Cardiac stimulation apparatus (10) such as a lead comprising one or more electrodes (17, 19 and 21) for electrical contact with heart or other tissue. An adhesive substance is provided for adhesively attaching the electrodes in position in relation to the heart or other tissue to provide a non-traumatic fixing procedure. The electrodes (17, 19, 21) are positioned on a support (15) to which the adhesive substance is applied attached.

This application is a Continuation-in-Part of application Ser. No. 10/297,189, filed 11 Jul. 2003, and which application(s) are incorporated herein by reference.

### FIELD OF THE INVENTION

This invention relates to a cardiac stimulation apparatus.

### BACKGROUND ART

There are occasions where it is necessary to apply electrical stimulation to the hearts of cardiac patients. Typically, electrical stimulation is applied using cardiac stimulation leads. Some cardiac stimulation leads are employed to apply electrical stimulation to the heart of a cardiac patient after surgical procedures to correct arrhythmic beating of the heart. The electrical stimulation may be applied to the atrium, to the ventricle, or sequentially to the atrium and the ventricle of the heart. Other electrical stimulation leads are used to apply electrical stimulation to the heart for the purpose of defibrillation.

Such leads employ an electrode or an electrode assembly at the distal end thereof through which electrical stimulation is applied. The electrode or electrode assembly is adapted to be positioned in electrical contact with the heart wall and retained in such position by a traumatic technique, such as by suturing a portion of the lead to the heart wall or threading a portion of the lead into the heart wall. Such techniques for retaining the electrode or electrode assembly in electrical contact with the heart wall can cause damage to the heart wall, not only during the attachment phase but possibly also during the subsequent detachment phase as the lead is withdrawn from the body of the patient by a pulling process.

It is against this background, and the difficulties associated therewith, that the present invention has been developed.

### DISCLOSURE OF THE INVENTION

According to one aspect of the present invention there is provided a cardiac stimulation apparatus comprising an electrode for electrical contact with heart or other tissue and means for adhesively attaching the electrode in position in relation to the heart or other tissue.

In this way there is no trauma to the heart or other tissue caused by the attachment process.

In one arrangement, the electrode may be attached directly by virtue of an adhesive substance on the electrode.

In another arrangement, the electrode may be attached indirectly by virtue of an adhesive substance on a support for the electrode.

The electrode may be a single electrode or it may be one of several electrodes in an electrode assembly.

Electrical stimulation may be delivered to the electrode by way of an electroconductive path from a source of electrical stimulation, in which case the stimulation apparatus may comprise a lead with the electrode provided at a distal end thereof and a proximal end thereof adapted to be connected to the source of electrical stimulation.

In another arrangement, electrical stimulation may be delivered to the electrode without the need for a lead to extend through the body of a patient undergoing cardiac stimulation to a source of electrical stimulation external to the body of the patient.

According to another aspect of the invention there is provided an cardiac stimulation lead comprising an electrode support at one end of the lead, first and second electrodes mounted in spaced relation on the electrode support, and a third electrode mounted on the electrode support in spaced relation to said first and second electrodes, said first and second electrodes being dimensioned for cardiac pacing and the third electrode having a larger electrical contact area than the first and second electrodes for cardiac defibrillation, and means for providing an electroconductive path between each electrode and the other end of the lead for electrical connection to a source of electrical stimulation.

Preferably, the third electrode has a large surface area and extends for at least a substantial part around the first and second electrodes.

The stimulation lead may be provided with a further electrode a larger electrical contact area than the first and second electrodes so that bipolar cardiac defibrillation can be performed.

Preferably, the electrode support comprises a wafer.

Preferably, the wafer is adapted to be placed against part of the heart or other tissue requiring stimulation with the necessary electrode or electrodes in electrical contact therewith.

For the purpose of maintaining the electrodes in electrical contact with the heart or other tissue, the wafer may be provided with an adhesive substance to bond it to the heart or other tissue.

Preferably, the wafer is laterally flexible for the purpose of conforming to the profile of that part of the heart or other tissue against which it is placed. For preference, the wafer has a dished configuration.

The stimulating electrode is intended to be temporarily placed in the body of a patient and is therefore preferably removable. A convenient way to remove the stimulating electrode is to simply pull it out of the body of the patient. To allow for removal in this manner, the wafer is preferably collapsible by virtue of its flexible nature to follow the course of the lead as it is drawn from the body of the patient.

On the other hand, the lead or at least part thereof may be constructed of biocompatible bio absorbable material, thereby eliminating the need to remove the lead from the body of the patient.

Preferably, the wafer is at one end of a flexible sheath which houses the electroconductive paths extending between the electrodes and the other end of the lead.

Preferably, the sheath is formed by twisting a ribbon of flexible material about the electroconductive paths.

Preferably, the electroconductive paths are established on the ribbon in electrically insulated relationship before twisting of the ribbon about its length.

The ribbon is preferably formed of the same material as the wafer and is integral therewith.

Preferably, connector means are provided at said other end of the stimulation lead for connection to the source of electrical stimulation, the electroconductive paths each being connected to a respective one of the connection means.

According to another aspect of the invention there is provided an epicardial stimulation lead comprising an elongated sheath, an electrode support at one end of the sheath and an electrode mounted on the electrode support, an electroconductive path passing through the sheath between the electrode and the other end of the sheath, characterised in that the sheath comprises a ribbon of flexible material twisted about its length.

Preferably, the electroconductive path is provided on the ribbon.

Preferably, the electrode support is integral with the ribbon.

According to still another aspect of the invention there is provided a method of making a stimulation lead comprising the steps of providing a ribbon of flexible material and an electrode support at one end of the ribbon, providing an electrode on the electrode support, forming an electroconductive path along the ribbon from the electrode to the other end of the ribbon, and twisting the ribbon about its length to create a sheath for housing the electroconductive path.

The electroconductive path may be of any suitable material such as a wire bonded onto the ribbon or a track of electroconductive material sprayed, printed or otherwise deposited on the ribbon.

There may be a plurality of electrodes supported on the electrode support and a plurality of electroconductive paths extending along the sheath. With such an arrangement, the electroconductive paths are electrically insulated from each other.

According to a still further aspect of the invention there is provided an epicardial stimulation electrode comprising an electrode support, an electrode on the electrode support, and an adhesive substance associated with the electrode for bonding the electrode to the heart or other tissue to maintain the electrode in electrical contact therewith.

According to a still further aspect of the invention there is provided a cardiac stimulation apparatus comprising an electrode and an electrical current generation means associated with the electrode for delivering an electrical current to the electrode when subjected to an external influence such as an energy wave.

The apparatus may further comprise an adhesive means for bonding the apparatus to head or other tissue with the electrode in electrical contact thereof.

According to yet another aspect of the invention there is provided a biomedical device comprising a substrate supporting an electrode, the substrate being attachable to internal tissue of a patient, the electrode being detachable from the substrate for removal from the patient while the substrate remains attached to the internal tissue, the substrate comprising a bioabsorbable material.

With this arrangement, it is not necessary for the substrate to be removed from the patient at the time of removal of the electrode and any lead attached thereto.

The substrate provides a platform on which the electrode is supported for electrical contact with internal tissue.

The substrate may support a single electrode or a plurality of electrodes.

Preferably, the substrate is provided with two forms of adhesion, a first form for initial adhesive contact with tissue and a second form for prolonged contact after the initial contact has been established.

The substrate may comprise several zones of adhesive, one providing the first form of adhesion and the other providing the second form of adhesion.

The substrate may comprise at least two layers, one layer providing a first zone of adhesion and another layer providing a second layer of adhesion. Typically, the layer proving the first zone of adhesion is located at one side of the substrate for immediate contact with the heart tissue.

Preferably, the biomedical device further comprises a lead having an electrical path connected to the electrode.

Where there is a plurality of electrodes, the lead may comprise a plurality of separate electrical paths, each connected respectively to one of the electrodes.

Preferably, the lead is detachably connected to the substrate.

Preferably, the lead is connected to the substrate by way of a detachable connection which can be detached upon pulling of the lead. In this way, pulling of the lead to effect its removal from the body of the patient causes separation of the lead and the electrode or electrode assembly connected thereto from the substrate, with the latter remaining attached to the internal tissue of the patient.

The detachable connection between the lead and the substrate may comprise a threaded connection therebetween; that is, the lead may be threaded through the substrate. Typically, the substrate may incorporate two holes through which the lead is threaded.

According to yet another aspect of the invention there is provided a biomedical device comprising a substrate attachable to internal tissue of a patent, a lead, and a connection for detachably connecting the substrate and the lead, whereby the lead can be detached from the substrate upon pulling of the lead.

With this arrangement, pulling of the lead to effect its removal from the body of the patient causes separation of the lead from the substrate, with the latter remaining attached to the internal tissue of the patient.

The detachable connection between the lead and the substrate may comprise a threaded connection therebetween; that is, the lead may be threaded through the substrate. Typically, the substrate may incorporate two holes through which the lead is threaded.

### BRIEF DESCRIPTION OF THE DRAWINGS

The invention will be better understood by reference to the following description of several specific embodiments thereof as shown in the accompanying drawings in which:

FIG. 1 is a plan view of an epicardial stimulation lead according to a first embodiment, shown in a condition prior to installation in the body of a cardiac patient;

FIG. 2 is a view of the stimulation lead according to the first embodiment, with the lead in a collapsed condition following withdrawn from the patient's body;

FIG. 3 is a view similar to FIG. 2 with the exception that the lead is now shown fitted with a threading needle used to facilitate threading of the lead through part of the body of the patient;

FIG. 4 is a plan view of the lead prior to twisting part of it to create a sheath;

FIG. 5 is a plan view of the lead according to a second embodiment prior to installation and showing a threading needle in position;

FIG. 6 is a collapsed view of an epicardial stimulation lead according to the second embodiment in a condition following withdrawn from a patient's body;

FIG. 7 is a plan view of the lead of the second embodiment prior to twisting to form a sheath;

FIG. 8 is a schematic view of a heart to which two stimulating electrodes according to the second embodiment have been attached;

FIG. 9 is a schematic plan view of a stimulation apparatus according to a third embodiment;

FIG. 10 is a cross-section along the line 10-10 of FIG. 9;

FIG. 11 is a schematic view of a heart to which two electrodes according to the third embodiment have been applied, one in association with the atrium and the other in association with the ventricle;

FIG. 12 is a schematic view illustrating a procedure for applying electrical stimulation utilising the two electrodes illustrating position in FIG. 11;

FIG. 13 is a schematic view of a heart to which two biomedical devices according to a fourth embodiment have been applied, one in association with the atrium and the other in association with the ventricle;

FIG. 14 is a view similar to FIG. 13 with the exception that the biomedical devices has been removed from the patient, with the result that each lead and the electrode assembly connected thereto have been removed and the substrate on which the electrode assembly was carried remains applied to the heart;

FIG. 15 is a fragmentary view of the biomedical device according to the embodiment of FIG. 13 illustrating the substrate at the distal end of the lead;

FIG. 16 is a view similar to FIG. 15 with the exception that the other side of the substrate is shown to illustrate the electrode assembly; and

FIG. 17 is a cross sectional view (in an exploded form) of the arrangement shown in FIG. 16.

### BEST MODE(S) FOR CARRYING OUT THE INVENTION

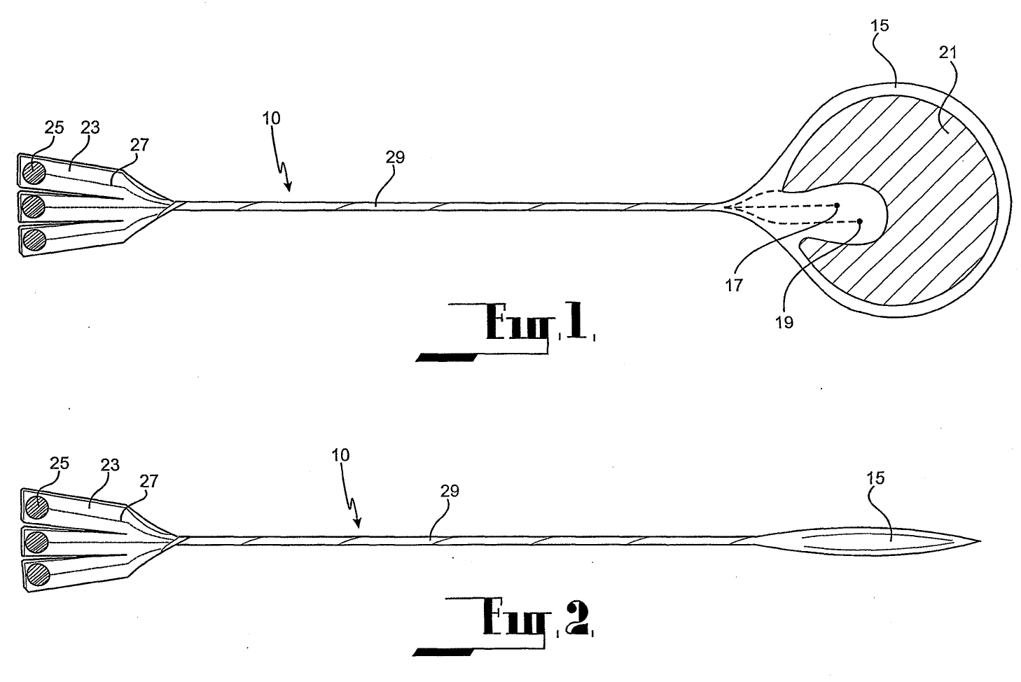

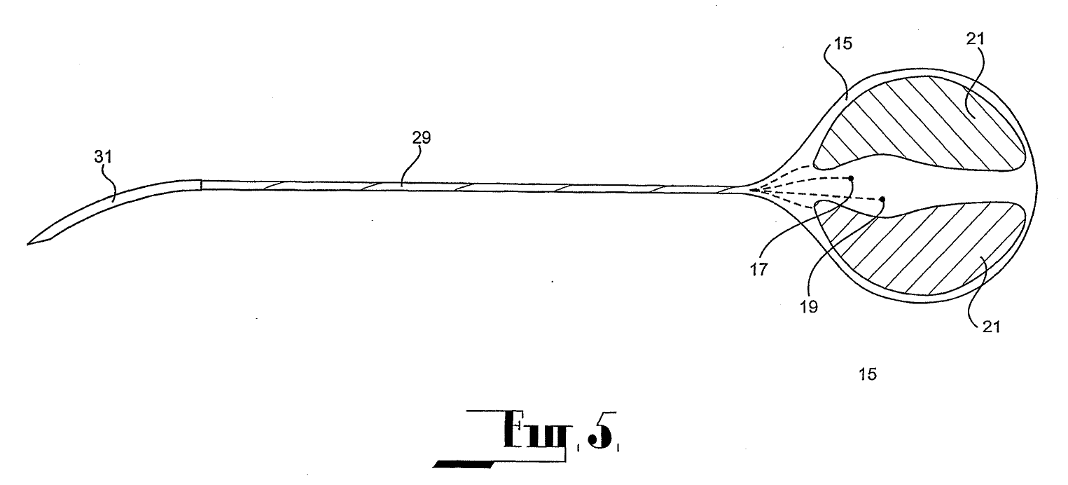

The embodiment shown in FIGS. 1 to 4 of the drawings is directed to an epicardial stimulation electrode which can be employed for either bipolar pacing or monopolar defibrillation of a heart.

The epicardial stimulating lead 10 comprises a length of polyethylene or other suitable material sheeting 11 of thickness up to approximately 30 microns which has been shaped to define a ribbon 13 and a wafer 15 of generally circular form at one end of the ribbon. The wafer 15 provides an electrode support on one face of which three electrodes are mounted, being first and second electrodes 17 and 19, and a third electrode 21, each spaced from the other. The first and second electrode 17 and 19 operate as a pair and are much smaller in terms of their electrically contact area than the third electrode 21, as can be seen from the drawings. Indeed, the electrical contact area of the third electrode far exceeds that of the first and second electrodes and it occupies a major part of the surface area of the side of the wafer 15 on which the electrodes are mounted.

The other end of the ribbon 13 branches into three sections 23 each of which supports an electrical terminal 25. Each electrical terminal 25 is connected to one of the electrodes by way of an electroconductive path 27.

The electroconductive paths 27 are provided on the ribbon and are electrically insulated from each other.

While the electrodes 17, 19 and 21, the terminals 25 and the electroconductive paths 27 may be of any suitable form, in this embodiment they have been deposited onto the sheet material by any suitable means such as printing or spraying.

The ribbon 13 is twisted along its length so as to create a sheath 29 which houses the electroconductive paths. The sheath 29 is laterally flexible by virtue of the flexible nature of the polyethylene.

The terminal end of the lead is adapted to receive a detachable needle 31 to facilitate threading of the lead through part of the body of a patient.

The wafer 15 is of dished configuration so that it conforms somewhat to a curved surface of the heart but is also sufficiently flexible to accommodate any variations which may be required to the preformed curvature. An adhesive substance (not shown) is applied to the wafer to facilitate bonding of the wafer to the heart with the electrodes in an electrical contact with the heart. The adhesive is biocompatible and electroconductive which ensures stable electroconductive contact between the electrodes and the heart.

The epicardial stimulation lead is intended to be temporarily implanted into the body of a cardiac patient before the pericardium and chest are closed. The wafer is placed in the required position against the heart according to the stimulation requirements and is maintained in such position by the adhesive bond. The sheath 29 is then fed through the pericardium and the thorax of the patient with the aid of the needle 31. Following penetration of the chest wall of the patient, the needle is detached from the sheath 29 so as to expose the terminals 25. The surgical procedure on the patient can then be completed as normal.

Should the heart of the patient require electrical stimulation in the post-operative period, it is merely necessary to connect a source of electrical stimulation to the appropriate terminals 25. If, for example, the patient requires arterial or ventrical pacing, an appropriate source of electrical stimulation is connected to the two terminals 25 which are coupled to the first and second electrodes 17 and 19. If, on the otherhand, defibrillation is required, an appropriate source of electrical stimulation is connected to the terminal 25 which is linked to the third electrode 21 these differing terminals being colour-coded. A return path for the electrical stimulation may be provided by either one of the electrodes 17 or 19, or a supplementary electrode attached to another part of the body of the patient.

As there is only one electrode sufficiently large enough for use in defibrillation procedures, the epicardial stimulating lead according to the first embodiment can only be employed for monopolar defibrillation.

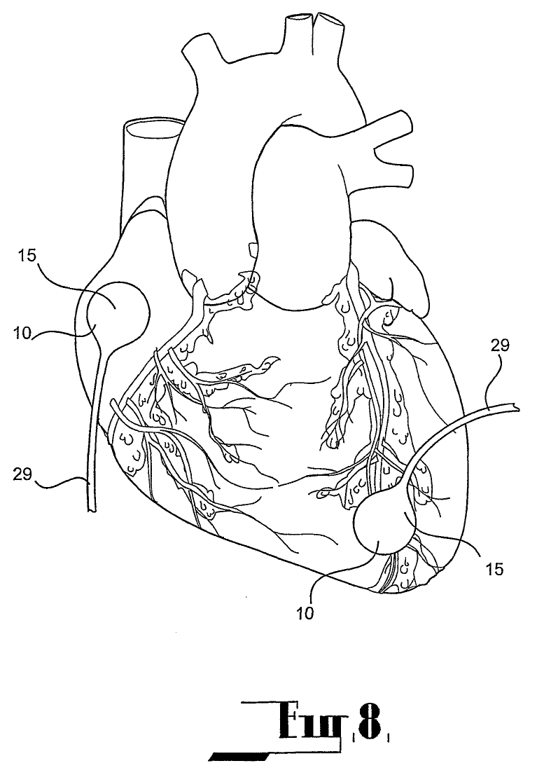

The epicardial stimulation lead according to the second embodiment, which is shown in FIGS. 5, 6 and 7 is similar to the first embodiment with the exception that there are four electrodes, being a pair of small electrodes 17 and 19 for use in cardiac pacing and a pair of large electrodes 21 for use in bipolar defibrillation.

When the epicardial stimulation lead according to either the first or second embodiment is no longer required in the body of the patient, it is simply pulled out through the chest wall. As the lead is pulled from the patient, the adhesive bond between the wafer and the heart separates and the wafer collapses upon itself as it follows the course of the lead outwardly through the body of the patient 15. The particular benefit of this arrangement is that there is little likelihood of damage to heart tissue as the wafer separates from the heart owing to the fact that it was only affixed to the heart by virtue of the mild adhesive bond.

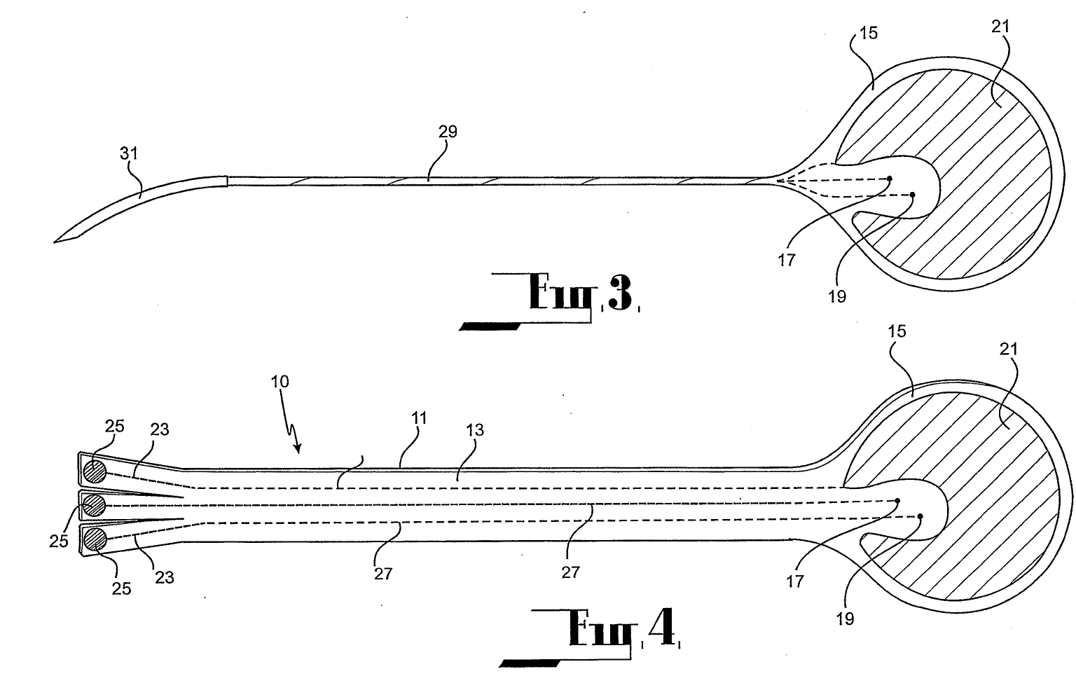

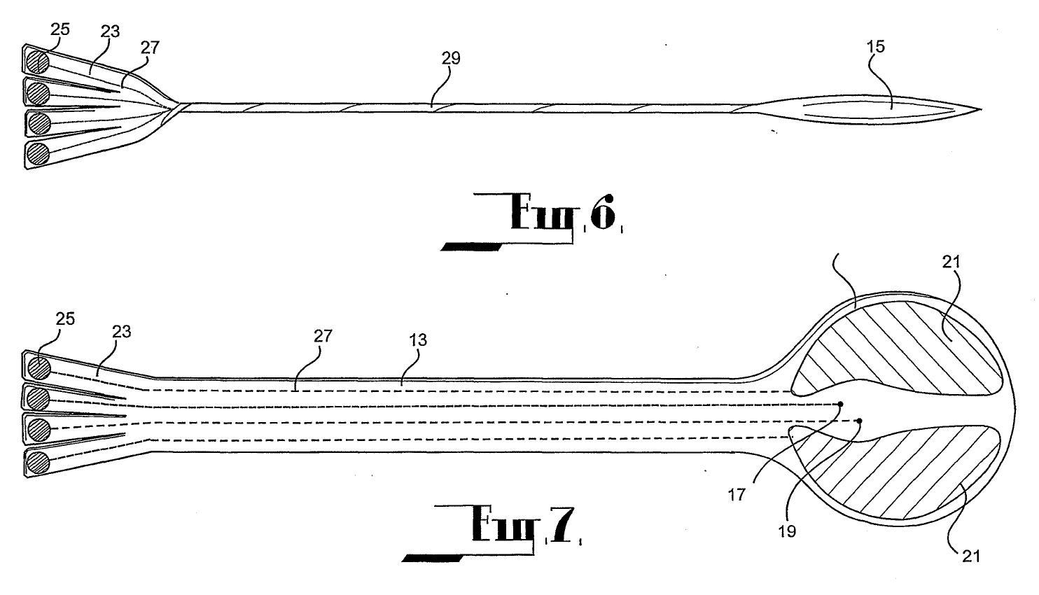

The collapsed condition of the wafer is illustrated in FIGS. 2 and 6.

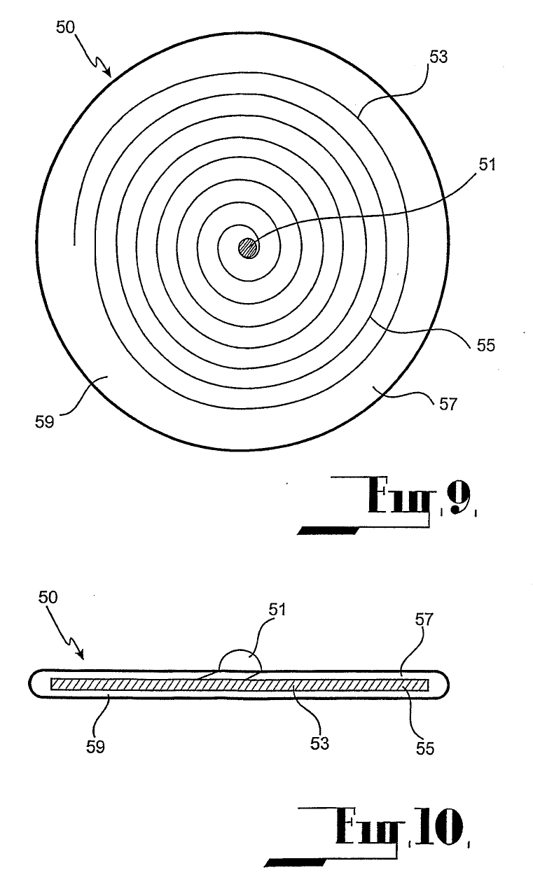

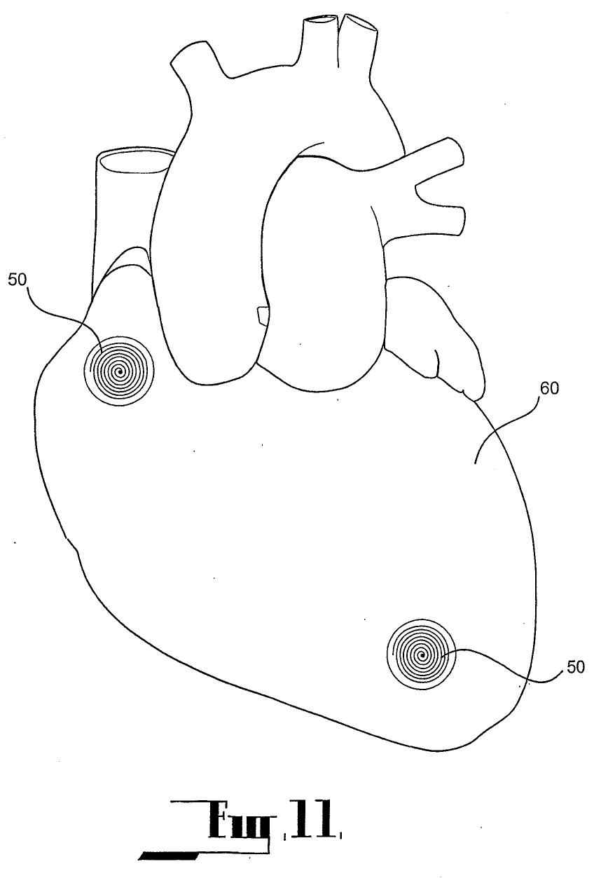

In the previous embodiments, the stimulation apparatus according to the invention is in the form of a lead, with an electrode or electrode assembly provided at a distal end thereof and the proximal end thereof being adapted to be connected to a source of electrical stimulation. Other arrangements are, of course, possible. One such prior arrangement is illustrated in FIGS. 9 to 12 of the accompanying drawings.

Referring now to FIGS. 9 to 12, there is shown a stimulation apparatus 50 comprising an electrode 51 and a means 53 for generating an electrical current for the electrode 51. In this embodiment, the current generation means 53 comprises an electroconductive coil 55 disposed about the electrode 51. The electrode 51 projects from the coil 55 (as shown in FIG. 10) to facilitate contact with the heart 60 or other tissue to which the apparatus 50 is applied.

The current generation means 53 is provided with adhesive means 57 by way of which the apparatus 50 can be adhesively bonded to the heart 60 or other tissue, with the electrode 51 in contact with the heart 60 or other tissue. In this embodiment, the adhesive means 57 is in the form of a biocompatible absorbable adhesive gel pad 59 positioned about the coil 55, with the electrode 51 extending beyond the gel pad 59.

The apparatus 50 is energised to generate an electrical current in the current generation means 53. In this embodiment, the apparatus 50 is energised by subjecting the coil 55 to an energy wave such as a magnetic wave or radio-frequency wave, with interaction between the coil 55 and energy wave being adapted to generate an electrical current which passes to the electrode 51.

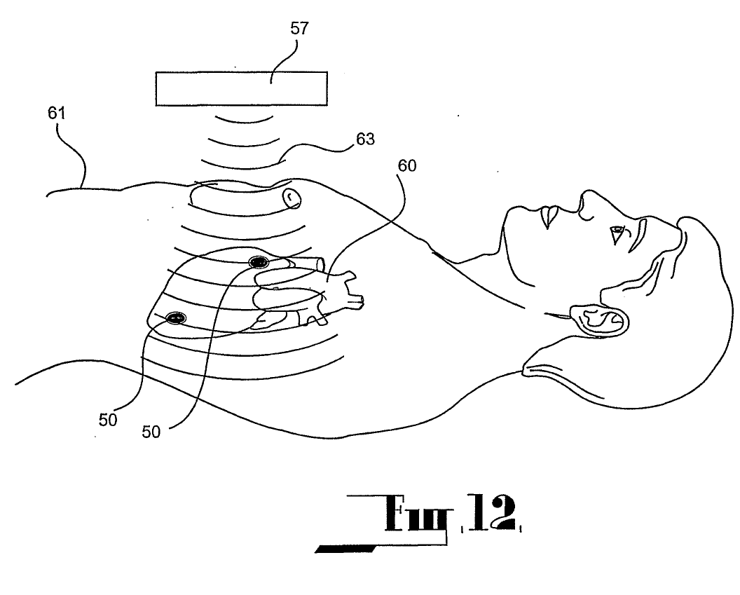

In this embodiment, the coil 55 is energised by an energy wave (depicted by lines 63) generated by a source 65 located exteriorly of the body 61 of the patient, as illustrated in FIG. 12 of the drawings.

The current path through the body 61 of the patient from the electrode 51 may be completed in any suitable fashion such as by provision of a further electrode, such as a skin electrode, positioned on an external portion of the body 61 of the patient.

A particular advantage of the apparatus according to this embodiment is that there is no lead which is passed through the body of the patient and which needs to be subsequently removed.

The embodiment shown in FIGS. 13 to 17 is directed to a biomedical device in the form of an epicardial stimulation lead assembly 100 which can be employed for either bipolar pacing or monopolar defibrillation of the heart.

The epicardial stimulation lead assembly 100 comprises a flexible lead 101 and an electrode assembly 103 connected to the lead 101 at the distal end 105 thereof. The proximal end (not shown) of the lead 101 is adapted for electrical connection to a cardiac machine.

In the arrangement shown, the electrode assembly 103 comprises two electrodes 107, 109. Each electrode, 107, 109 is electrically connected to an electrode conductive path 111 in the form of a wire. The two wires are electrically insulated from each other and are encased within, and extend along, the lead 101.

The electrode assembly 103 is supported on a substrate 115. With this arrangement, the substrate 115 provides a platform for the two electrodes 107, 109. The substrate 115 is adapted to be adhesively attached to heart tissue, with the electrodes 107, 109 in electrical contact with the heart tissue.

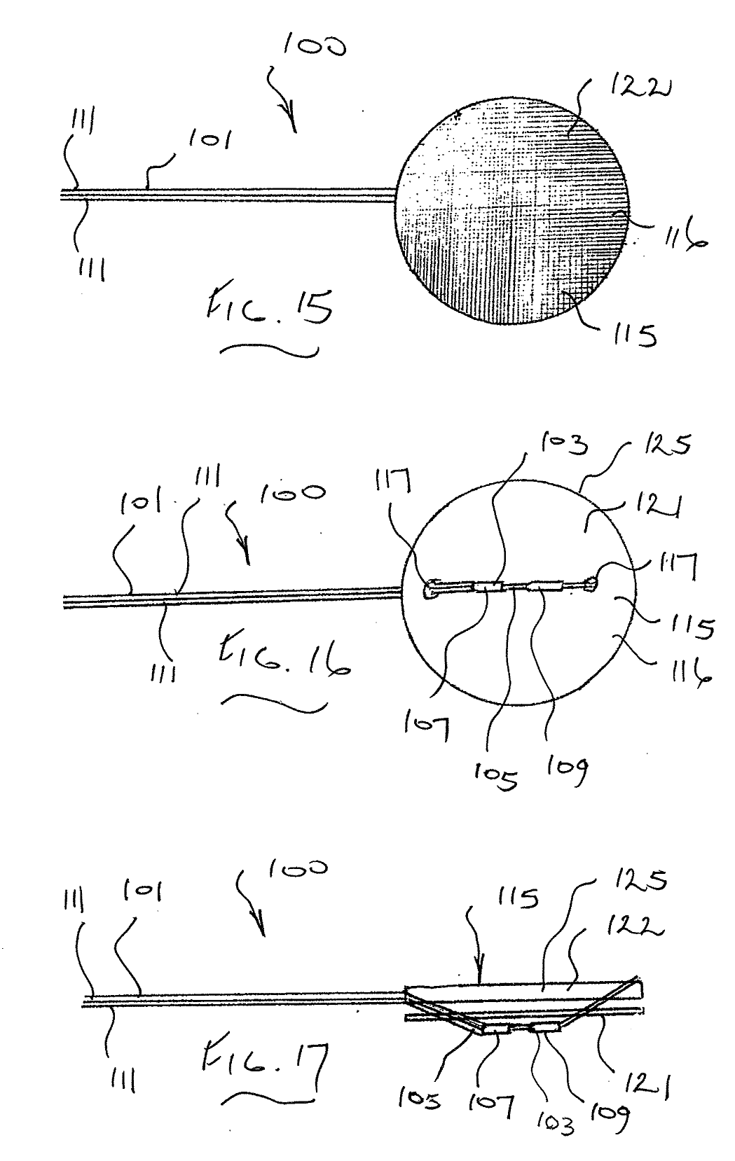

The electrodes, 107, 109, as well as the electrical lead 101, are detachably connected to the substrate 115 by being releasably attached thereto. The arrangement is such that the electrodes 107, 109 can separate from the substrate 115 upon pulling of the lead 101 once the substrate 115 is adhesively attached to the heart tissue. In the arrangement shown, the distal end 105 of the lead 101 is threaded through the substrate 115, thereby allowing the lead 101 and the electrode assembly 103 connected thereto, to separate from the substrate 115 upon pulling of the lead 101. For this purpose, the substrate 115 incorporates two holes 117 through which the distal end 105 of the lead 101 is threaded, as shown in FIGS. 16 and 17. When the distal end 105 of the lead 101 is threaded through the substrate, the electrode assembly 103 is exposed for presentation to heart tissue to which the substrate 115 is attached. With this threaded connection, the distal end 105 of the lead can be separated from the substrate 115 by movement in the reverse direction with respect to the substrate; that is, by pulling the distal end 105 in the direction opposite to that in which it was threaded into the substrate 115.

The substrate 115 comprises an adhesive gel pad 116 formed of biocompatible, biodegradable materials. In the arrangement shown, the substrate is of composite construction comprising first layer 121 and a second layer 122. In another arrangement, the adhesive gel pad 116 may be of unitary construction.

The substrate 115 is absorbable within the body of the patient over time after completing it function as part of the epicardial stimulation lead assembly 100. Accordingly, the two layers 121, 122 are made of appropriate bioabsorbable materials.

The first layer 121 is the innermost layer and is adapted to confront heart tissue to which the substrate 115 is applied.

The second layer 122 comprises a long-lasting adhesive capable of adhering to the heart tissue of a cardiac surgery patient for several days post surgery and provides a base 125 on which the electrode assembly 103 is supported. The base 125 incorporates the two holes 117 through which the distal end 105 of the lead 101 is threaded, as previously described. Any appropriate biocompatible material may be utilised for the base 125, typical examples of which are fibrin, collagen, hyaluronic acid, acrylic acid or other such biocompatible biodegradable materials and combinations thereof.

The first layer 121 comprises a thin coating of a rapid-action adhesive which is applied to the adjacent face of the base 125. The rapid-action adhesive is intended to provide immediate adhesive contact to heart tissue to which it is applied. An appropriate biodegradable, biocompatible fast-acting adhesive may be utilised for the first layer 121. It may be made from similar materials to the base 125 but formulated to be a rapid-acting adhesive.

Use of the epicardial stimulation lead assembly 100 will now be described. The substrate 115 is applied to the heart 130 of the patient with the electrode assembly 103 in electrical contact therewith. The substrate 115 is attached to the heart tissue adhesively by presenting the first layer 121 to the heart tissue. This rapidly establishes an adhesive contact with the heart tissue, so retaining the electrode assembly 103 in contact with the heart tissue initially. The initial adhesive contact established by layer 121 is later supplemented, and ultimately supplanted, by the adhesive characteristics of the second layer 122.

Once the substrate 115 is in adhesive contact with the heart tissue (by way of the initial adhesion provided by the first layer 121), the lead 101 can be threaded through the abdominal wall of the patient in accordance with known procedures and the proximal end thereof (not shown) connected to an appropriate cardiac machine, as required. In the arrangement shown in FIG. 13, two epicardial stimulation lead assemblies 100a and 100b have been implanted in the patient and are shown attached to the heart 130.

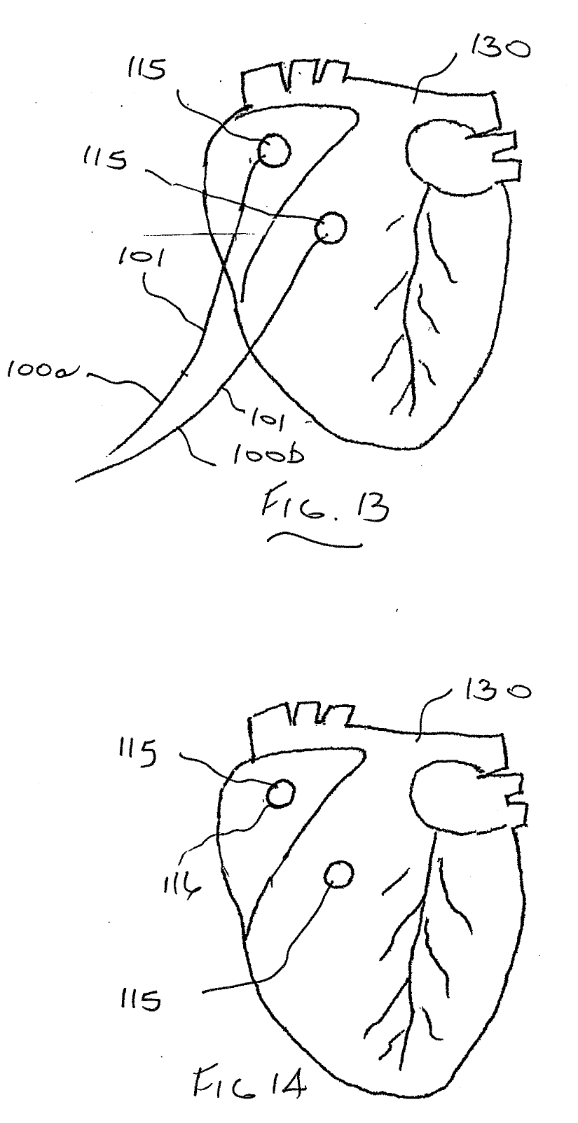

When the cardiac stimulation is no longer necessary, the two epicardial stimulation lead assemblies 100a and 100b can be removed. This is done by pulling the lead 101 of each one of the two epicardial stimulation lead assemblies 100a and 100b outwardly through the abdominal wall of the patient. The application of the pulling force to each lead 101 causes the lead, as well as the electrode assembly 103 connected to the lead, to separate from the substrate 115 in adhesive contact with the heart tissue. The substrate 115 remains attached to the heart tissue (as shown in FIG. 14), while the lead 101 and the electrode assembly 103 are withdrawn from the patient. In this way, the lead 101 and the electrode assembly 103 are removed from the body patient but the substrate 115 remains in place. It is unnecessary to remove the substrate 115 as part of the procedure as it will over time be absorbed and excreted by natural processes of the body of the patient.

From the foregoing, it is evident that this fourth embodiment provides a simple yet highly effective arrangement for implantation in, and removal of, an epicardial stimulation lead assembly with respect to a body of a cardiac patient.

It should be appreciated that the invention is not limited to the epicardial stimulation lead assembly according to the embodiments described. The invention may be applied to any biomedical device for transferring electrical impulses to the cardiac system of a person. In addition to cardiac stimulation the invention may possibly have application in cardiac monitoring.

Modifications and improvements may be made without departing from the scope of the invention.

Throughout the specification, unless the context requires otherwise, the word “comprise” or variations such as “comprises” or “comprising”, will be understood to imply the inclusion of a stated integer or group of integers but not the exclusion of any other integer or group of integers.

The claims defining the invention are as follows:

1. Cardiac stimulation apparatus comprising an electrode support, an electrode provided on the support for electrical contact with internal tissue within the body of a patient, and means for releasably attaching the support in position in relation to the internal tissue, an electrical lead defining an electroconductive path for delivering stimulation to the electrode, the support being forcibly removable from the internal tissue and also collapsible upon application of a pulling force to the electrical lead to facilitate withdrawal of the support from the body of the patient along with the electrical lead.

2. A cardiac stimulation apparatus according to claim 1 wherein the support is releasably attachable by adhesive attachment comprising an adhesive substance on the support for the electrode.

3. A cardiac stimulation apparatus according to claim 2 wherein the support comprises a wafer.

4. A cardiac stimulation apparatus according to claim 2 wherein the support comprises an adhesive gel pad.

5. A cardiac stimulation apparatus according to claim 1 wherein the electrode is provided at a distal end thereof and a proximal end thereof adapted to be connected to the source of electrical stimulation.

6. A cardiac stimulation lead comprising an electrode support at one end of the lead, first and second electrodes mounted in spaced relation on the electrode support, and a third electrode mounted on the electrode support in spaced relation to said first and second electrodes, said first and second electrodes being dimensioned for cardiac pacing and the third electrode having a larger electrical contact area than the first and second electrodes for cardiac defibrillation, and means for providing an electroconductive path between each electrode and the other end of the lead for electrical connection to a source of electrical stimulation, means for releasably attaching the support in position in relation to internal tissue within the body of a patient, the support being forcibly removable from the internal tissue and also collapsible upon application of a pulling force to the cardiac stimulation lead to facilitate withdrawal of the support from the body of the patient.

7. A cardiac stimulation lead according to claim 6 wherein the third electrode has a large surface area and extends for at least a substantial part around the first and second electrodes.

8. A cardiac stimulation lead according to claim 6 further comprising a further electrode having a larger electrical contact area than the first and second electrodes so that bipolar cardiac defibrillation can be performed.

9. A cardiac stimulation lead according to claim 6 wherein the electrode support comprises a wafer.

10. A cardiac stimulation lead according to claim 9 wherein the wafer is adapted to be placed against part of the heart or other tissue requiring stimulation with the necessary electrode or electrodes in electrical contact therewith.

11. A cardiac stimulation lead according to claim 9 wherein the wafer is provided with an adhesive substance to bond it to the heart or other tissue.

12. A cardiac stimulation lead according to claim 9 wherein the wafer is laterally flexible for the purpose of conforming to the profile of that part of the heart or other tissue against which it is placed.

13. A cardiac stimulation lead according to claim 9 wherein the wafer is of a dished configuration.

14. A cardiac stimulation lead according to claim 9 wherein the wafer is at one end of a flexible sheath which houses the electroconductive path extending between the or each electrode and the other end of the lead.

15. A cardiac stimulation lead according to claim 14 wherein the sheath is formed by twisting a ribbon of flexible material about the electroconductive path.

16. A cardiac stimulation lead according to claim 15 wherein the or each electroconductive path is established on the ribbon in electrically insulated relationship before twisting of the ribbon about its length.

17. A cardiac stimulation lead according to claim 15 wherein the ribbon is of the same material as the wafer and is integral therewith.

18. An epicardial stimulation lead, comprising: an elongated sheath, an electrode support at one end of the sheath and an electrode mounted on the electrode support, an electroconductive path passing through the sheath between the electrode and the other end of the sheath, wherein the sheath comprises a ribbon of flexible material twisted about its length.

19. An epicardial stimulation lead according to claim 18 wherein the electroconductive path is provided on the ribbon.

20. An epicardial stimulation lead according to claim 18 wherein the electrode support is integral with the ribbon.

21. A method of making a stimulation lead comprising the steps of providing a ribbon of flexible material and an electrode support at one end of the ribbon, providing an electrode on the electrode support, forming an electroconductive path along the ribbon from the electrode to the other end of the ribbon, and twisting the ribbon about its length to create a sheath for housing the electroconductive path.

22. A biomedical device comprising a substrate supporting an electrode, the substrate being attachable to internal tissue of a patient, the electrode being detachable from the substrate for removal from the patient while the substrate remains attached to the internal tissue, the substrate comprising a bioabsorbable material.

23. A biomedical device according to claim 22 wherein the substrate comprises an adhesive gel pad.

24. A biomedical device according to claim 22 wherein the substrate has two forms of adhesion, a first form for initial adhesive contact with tissue and a second form for prolonged contact after the initial contact has been established.

25. A biomedical device according to claim 24 wherein the substrate comprises several zones of adhesive, one providing the first form of adhesion and the other providing the second form of adhesion.

26. A biomedical device according to claim 25 wherein the substrate comprises two layers, one layer providing a first zone of adhesion and another layer providing a second layer of adhesion.

27. A biomedical device according to claim 22 further comprising a lead having an electrical path connected to the electrode.

28. A biomedical device according to claim 27 wherein the lead is detachably connected to the substrate.

29. A biomedical device according to claim 28 wherein the lead is connected to the substrate by way of a connection detachable upon pulling of the lead.

30. A biomedical device according to claim 29 wherein the detachable connection between the lead and the substrate comprises a threaded connection therebetween.

31. A biomedical device according to claim 30 wherein the substrate comprises two holes through which the lead is threadable.
